(19) United States
(12) Patent Application Publication
(10) Pub. No.: US 2018/0028107 A1
(43) Pub. Date: Feb. 1, 2018

(54) SKIN DIAGNOSIS DEVICE

(71) Applicant: PANASONIC INTELLECTUAL PROPERTY MANAGEMENT CO., LTD., Osaka (JP)

(72) Inventors: Koji HORIUCHI, Fukuoka (JP); Hiroto TOMITA, Fukuoka (JP)

(73) Assignee: PANASONIC INTELLECTUAL PROPERTY MANAGEMENT CO., LTD., Osaka (JP)

(21) Appl. No.: 15/548,153

(22) PCT Filed: Feb. 2, 2016
(86) PCT No.: PCT/JP2016/000516

§ 371 Date: Aug. 2, 2017

(30) Foreign Application Priority Data
Mar. 26, 2015 (JP) 2015-064060

(51) Int. Cl. A61B 5/00

(52) U.S. Cl. CPC A61B 5/441; A61B 5/0077; A61B 5/7282; A61B 2576/00; A61B 2562/0233

(57) ABSTRACT

A skin diagnosis device includes an imaging unit (17) that images a face of a person who is subjected to a diagnosis, a facial part analyzer (18) that analyzes a facial part, based on a facial image of the person who is subjected to the diagnosis, an irradiation management unit (13) that specifies positions of eyes of the person who is subjected to the diagnosis from an analysis result of the facial part, and determines a whole of a facial region except for the eye positions as an irradiation region, an irradiation unit (15) that irradiates the determined irradiation region with light, and a skin diagnosis analyzer (19) that performs a skin diagnosis, based on the facial image, which is imaged by being irradiated with the light, of the person who is subjected to the diagnosis. It is possible to protect the eyes of the person who is subjected to the diagnosis, and to relieve stress of the person who is subjected to the diagnosis.

### TECHNICAL FIELD

The present disclosure relates to a skin diagnosis device.

### BACKGROUND ART

In a skin diagnosis device that diagnoses a skin condition such as a spot, dullness, or a wrinkle of a face, there are a contact type and a non-contact type. A sensor and a lighting are placed in the contact type skin diagnosis device, and the contact type skin diagnosis device photographs the skin condition by directly being into contact with the skin. Therefore, in the contact type skin diagnosis device, there is a need to perform measurements as many as the number of diagnosis positions, and the number of operations by an operator are large.

On the contrary, the non-contact type skin diagnosis device irradiates a whole of the face with light, and photographs the whole of the face at one time (for example, see PTL 1). Therefore, in the non-contact type skin diagnosis device, it is possible to analyze each diagnosis position by the photographing of one time, and the number of operations by the operator become small.

However, in the non-contact type skin diagnosis device, since the irradiation is performed with the lighting having high illuminance, it is not desirable to photograph a person who is subjected in a diagnosis in a state where the person who is subjected to the diagnosis opens eyes. Moreover, the person who is subjected to the diagnosis feels dazzling even if the eyes are closed, and feels stress that the eyes be necessarily closed.

Objects of the present disclosure are to protect the eyes of the person who is subjected to the diagnosis, and to relieve the stress of the person who is subjected to the diagnosis.

### CITATION LIST

### Patent Literature

PTL 1: Japanese Patent Unexamined Publication No. 2004-105748

### SUMMARY OF THE INVENTION

According to an aspect of the present disclosure, there is provided a skin diagnosis device including an imaging unit that images a face of a person who is subjected to a diagnosis, a facial part analyzer that analyzes a facial part, based on a facial image of the person who is subjected to the diagnosis, an irradiation management unit that specifies positions of eyes of the person who is subjected to the diagnosis from an analysis result of the facial part, and determines a whole of a facial region except for the eye positions as an irradiation region, an irradiation unit that irradiates the determined irradiation region with light, and a skin diagnosis analyzer that performs a skin diagnosis, based on the facial image, which is imaged by being irradiated with the light, of the person who is subjected to the diagnosis.

According to the present disclosure, it is possible to protect the eyes of the person who is subjected to the diagnosis, and to relieve stress of the person who is subjected to the diagnosis.

### BRIEF DESCRIPTION OF DRAWINGS

FIG. 1 is a block diagram illustrating a configuration of a skin diagnosis device according to an exemplary embodiment.

FIG. 2 is a diagram illustrating a management table which is provided with an irradiation management unit.

FIG. 3 is a schematic diagram illustrating a state where an irradiation unit performs irradiation with respect to a face of a person who is subjected to a diagnosis.

FIG. 4 is a diagram illustrating a display example of a course selection screen.

FIG. 5 is a diagram illustrating a display example of an analysis part selection screen.

FIG. 6 is a flowchart illustrating a processing procedure of the skin diagnosis device illustrated in FIG. 1.

FIG. 7 is a schematic diagram illustrating a state where the irradiation unit performs the irradiation partially with respect to the face of the person who is subjected to the diagnosis.

### DESCRIPTION OF EMBODIMENTS

Hereinafter, exemplary embodiments of the present disclosure will be described in detail, with reference to the drawings.

### Exemplary Embodiment

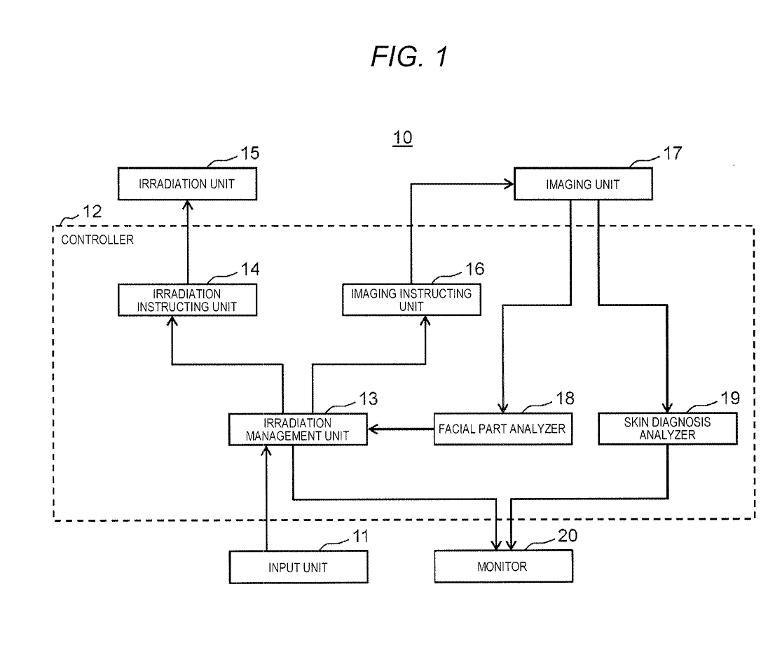

FIG. 1 is a block diagram illustrating a configuration of skin diagnosis device 10 according to an exemplary embodiment. Hereinafter, the configuration of skin diagnosis device 10 will be described, by using FIG. 1.

Input unit 11 is an interface that receives an input of operation information by an operator, and for example, input unit 11 is at least one of a keyboard, a mouse, a microphone, a touch panel, and the like. Specifically, input unit 11 outputs an analysis item (such as a pore, a spot, a wrinkle, moisture content, a nasolabial fold, or a texture) and an analysis part (such as a nose, a cheek, or an eye area) of a skin which are input by the operator to irradiation management unit 13.

Controller 12 includes irradiation management unit 13, irradiation instructing unit 14, imaging instructing unit 16, facial part analyzer 18, and skin diagnosis analyzer 19.

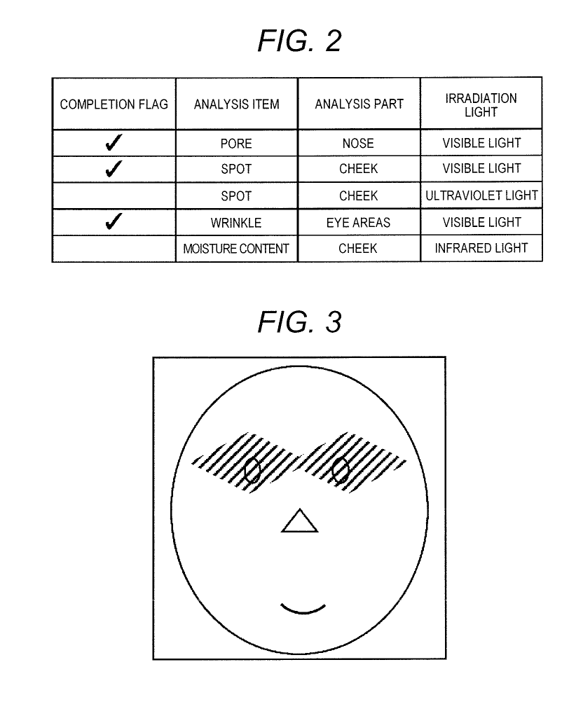

Irradiation management unit 13 determines appropriate irradiation light from visible light, ultraviolet light, infrared light, and the like, depending on the analysis item which is input from input unit 11, and notifies irradiation instructing unit 14 of the determined irradiation light. Irradiation management unit 13 specifies positions of eyes of a person who is subjected to a diagnosis, by an analysis result which is output from facial part analyzer 18, and determines a whole of a facial region except for the eye positions as an irradiation region, and notifies irradiation instructing unit 14 of the determined irradiation region. Irradiation management unit 13 notifies imaging instructing unit 16 of photographing timing which is input from input unit 11. Irradiation management unit 13 includes a management table as illustrated in FIG. 2, and manages whether or not a skin diagnosis analysis is completed per combination of the analysis item, the analysis part, and the irradiation light by a completion flag. In the combination in which the analysis can be performed at one time, all analyses are performed at one time, but in a case where all analyses are not performed at one time such that the analysis parts are overlapped with respect to the analysis items which are different from each other, irradiation management unit 13 does not set up the completion flag with respect to the combination corresponding thereto. Irradiation management unit 13 instructs such that the skin diagnosis analyses are repeatedly performed until the completion flags are set up with respect to all combinations which are input by the operator. Irradiation management unit 13 outputs a selection screen onto monitor 20.

Irradiation instructing unit 14 instructs irradiation unit 15 to obtain the irradiation region and the irradiation light which are notified from the irradiation management unit 13.

For example, irradiation unit 15 is a projector, and irradiates the irradiation region which is instructed in the same manner, with the irradiation light which is instructed by irradiation instructing unit 14. As illustrated in FIG. 3, irradiation unit 15 performs the irradiation of the whole of the facial region except for the eyes of the person who is subjected to the diagnosis. FIG. 3 illustrates that the irradiation is not performed in a half-tone dot meshing region including the eyes. Thereby, it is possible to protect the eyes of the person who is subjected to the diagnosis.

Imaging instructing unit 16 instructs imaging unit 17 to perform imaging at imaging timing which is notified from irradiation management unit 13.

Imaging unit 17 performs the imaging at the timing which is instructed by imaging instructing unit 16, and outputs the imaged image to facial part analyzer 18 or skin diagnosis analyzer 19.

Facial part analyzer 18 analyzes the facial part such as the eye, the nose, a mouth, a forehead, or the cheek, based on the facial image which is output from imaging unit 17, and outputs the analysis result to irradiation management unit 13.

Skin diagnosis analyzer 19 performs a skin diagnosis, based on the image which is output from imaging unit 17, and outputs a skin diagnosis result to monitor 20.

Monitor 20 displays character information and image information. Specifically, monitor 20 displays the selection screen, the skin diagnosis result which is output from skin diagnosis analyzer 19, and the like.

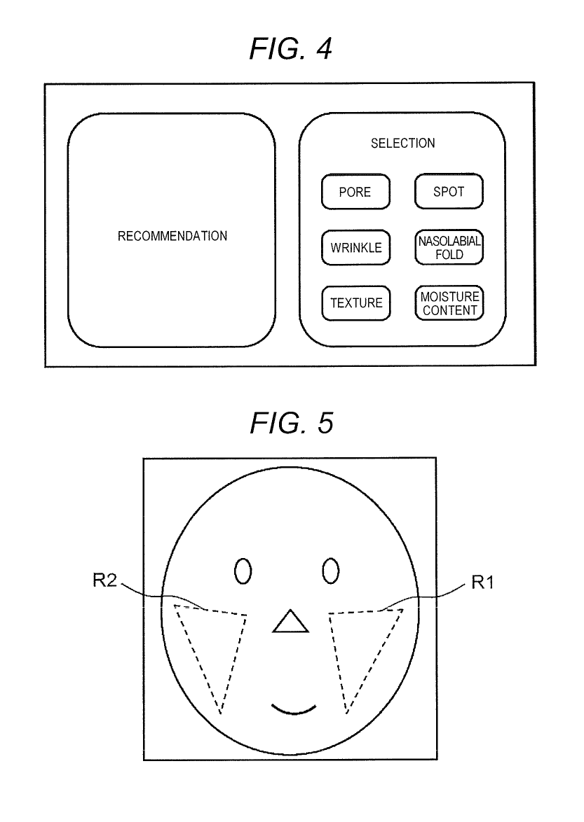

Here, examples of the selection screens which are displayed on monitor 20 are illustrated in FIG. 4 and FIG. 5. FIG. 4 illustrates that it is possible to select a recommendation course and a selection course. The recommendation course is a course in which the analysis item and the analysis part are automatically determined, and the selection course is a course in which the analysis item and the analysis part are designated by the operator. FIG. 5 illustrates a state where the operator selects the cheek of the person who is subjected to the diagnosis, among the selectable analysis parts. Regions R1 and R2 which are indicated by frames of dotted lines in FIG. 5 illustrate a case where the cheeks are selected.

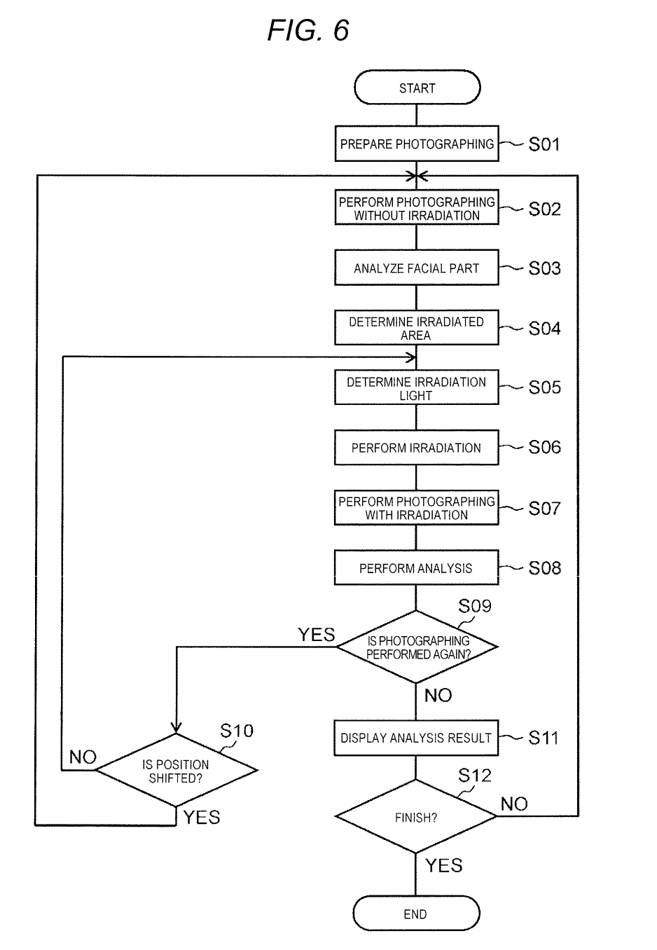

Next, a processing procedure of skin diagnosis device 10 illustrated in FIG. 1 will be described by using FIG. 6. FIG. 6 is a flowchart illustrating the processing procedure of skin diagnosis device 10 illustrated in FIG. 1.

In step S01, skin diagnosis device 10 performs imaging preparation such that software is activated, by the operation of the operator, and in step S02, imaging unit 17 images the whole of the face of the person who is subjected to the diagnosis without the irradiation from the irradiation unit 15.

In step S03, facial part analyzer 18 analyzes the facial part from the facial image which is imaged in step S02, and in step S04, irradiation management unit 13 determines the whole of the facial region except for the positions of the eyes as an irradiation region, based on the facial part which is analyzed in step S03.

In step S05, irradiation management unit 13 determines the appropriate irradiation light, depending on the analysis item, and in step S06, irradiation unit 15 irradiates the irradiation region which is determined in step S04 with the irradiation light which is determined in step S05.

In step S07, imaging unit 17 images the face, on which the irradiation is performed from irradiation unit 15, of the person who is subjected to the diagnosis, and in step S08, skin diagnosis analyzer 19 analyzes the facial image which is imaged in step S07.

In step S09, in a case where the analysis may not be performed at one time such that the analysis parts are overlapped with respect to the analysis items which are different from each other, or the irradiation lights are different from each other depending on the analysis items, imaging unit 17 determines whether or not to image the face of the person who is subjected to the diagnosis again. In a case where the imaging is performed again (step S09: YES), the processing proceeds to step S10, and in a case where the imaging is not performed again (step S09: NO), the processing proceeds to step S11.

In step S10, facial part analyzer 18 determines whether or not a position of the face is shifted from a predetermined position due to lapse of a predetermined time or the like, and in a case where the position is shifted (step S10: YES), the processing returns to step S02, and in a case where the position is not shifted (step S10: NO), the processing returns to step S05.

In step S11, monitor 20 displays the analysis result which is obtained in step S08, and in step S12, irradiation management unit 13 determines whether or not a finish instruction is received from input unit 11, and in a case where the finish instruction is received (step S12: YES), the processing of skin diagnosis device 10 is finished, and in a case where the finish instruction is not received (step S12: NO), the processing returns to step S02.

In this manner, according to the present exemplary embodiment, the skin diagnosis analyzer specifies the positions of the eyes of the person who is subjected to the diagnosis, based on the facial image, which is imaged without performing the irradiation with the light, of the person who is subjected to the diagnosis, and determines the whole of the facial region except for the eyes of the person who is subjected to the diagnosis as an irradiation region, and determines the irradiation light depending on the analysis item, and irradiates the determined irradiation region with the determined irradiation light, and thereby, it is possible to protect the eyes of the person who is subjected to the diagnosis, and to relieve stress of the person who is subjected to the diagnosis.

### Modification Example

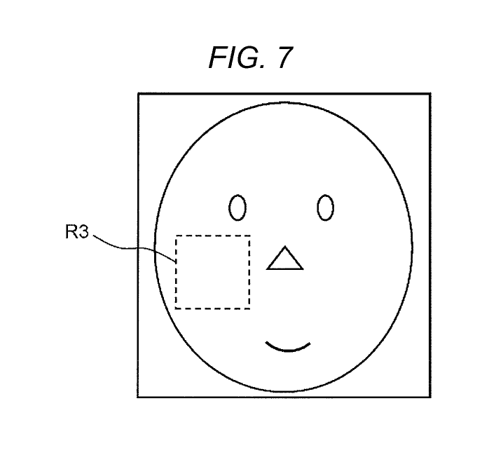

In the exemplary embodiment described above, it is described that irradiation management unit 13 determines the whole of the facial region except for the eyes of the person who is subjected to the diagnosis as an irradiation region, but the present invention is not limited thereto. That is, as illustrated in FIG. 7, irradiation management unit 13 may determine a fixed area including the analysis part as irradiation region R3. This is because a feature within the fixed area, for example, pore density or the like is used as an index, in the skin diagnosis. As a fixed area, it is desirable that a region where unevenness of the face is relatively small, such as the cheek or the forehead, is used.

### INDUSTRIAL APPLICABILITY

The skin diagnosis device according to the present disclosure is useful to protect the eyes of the person who is subjected to the diagnosis, and to relieve the stress of the person who is subjected to the diagnosis.

### REFERENCE MARKS IN THE DRAWINGS

10 skin diagnosis device
11 input unit
12 controller
13 irradiation management unit
14 irradiation instructing unit
15 irradiation unit
16 imaging instructing unit
17 imaging unit
18 facial part analyzer
19 skin diagnosis analyzer
20 monitor

1. A skin diagnosis device comprising:
an imaging unit that images a face of a person who is subjected to a diagnosis;
a facial part analyzer that analyzes a facial part, based on a facial image of the person who is subjected to the diagnosis;
an irradiation management unit that specifies positions of eyes of the person who is subjected to the diagnosis from an analysis result of the facial part, and determines a whole of a facial region except for the eye positions as an irradiation region;
an irradiation unit that irradiates the determined irradiation region with light; and
a skin diagnosis analyzer that performs a skin diagnosis, based on the facial image, which is imaged by being irradiated with the light, of the person who is subjected to the diagnosis.

2. The skin diagnosis device of claim 1,
wherein the skin diagnosis analyzer performs the skin diagnosis at one time with respect to a plurality of analysis items.

3. The skin diagnosis device of claim 1,
wherein the irradiation management unit determines irradiation light having a wavelength which is suitable for the analysis item, and
the irradiation unit performs irradiation with the irradiation light having the determined wavelength.

4. The skin diagnosis device of claim 1,
wherein the irradiation management unit determines a fixed area including an analysis part as an irradiation region, and
the irradiation unit irradiates the determined irradiation region with the irradiation light.

5. The skin diagnosis device of claim 1,
wherein the irradiation unit is a projector.
